(19) United States
(12) Patent Application Publication
(10) Pub. No.: US 2022/0330493 A1
(43) Pub. Date: Oct. 20, 2022

(54) SYSTEM, METHOD AND APPARATUS FOR CREATING LAMINAR AIR FLOW FOR INDOOR GROWING ENVIRONMENTS

(71) Applicant: CANNAFLOW AIR SYSTEM INC., Monrovia, CA (US)

(72) Inventor: Markus Kashinsky, Monrovia, CA (US)

(21) Appl. No.: 17/725,443

(22) Filed: Apr. 20, 2022

Related U.S. Application Data
(60) Provisional application No. 63/177,306, filed on Apr. 20, 2021.

(51) Int. Cl. A01G 9/24; A61L 9/22

(52) U.S. Cl. CPC A01G 9/246; A61L 9/22; A61L 2209/16

(57) ABSTRACT

A system, method and apparatus for creating laminar air flow inside an indoor growing environment is described. Ambient air inside the indoor growing environment is drawn into one or more fan casings coupled to an air intake manifold. The ambient air is then expelled from the air intake manifold, through two or more air outlet ports and into to or more air tubes, respectively. Each of the air tubes comprises a plurality of air tube orifices that are sized and positioned along each of the plurality of air tubes to expel the ambient air inside the air tubes in such a way as to create a laminar air flow through plants arranged on either side of each of the air tubes.

### I. CROSS-REFERENCE TO RELATED APPLICATIONS

This application claims the benefit of U.S. provisional patent application Ser. No. 63/177,306, filed on Apr. 20, 2021.

### II. FIELD

This present invention generally relates to airflow engineering, and more particularly to laminar airflow systems for indoor growing environments.

### III. BACKGROUND

Indoor plant farms are highly dependent on environmental control to maintain precise temperatures, humidity, and other conditions to promote optimal plant growth. However, it is difficult to maintain a consistent air quality throughout grow rooms, especially ones that utilize multiple growing tiers. Multi-tier grow rooms are popular because growers can increase the number of plants without increasing the square footage of a grow room. Such multi-tier grow rooms may be found in dense, urban areas because they require less square footage than traditional greenhouses. In such multi-tiered growing environments, shelving is used to form the tiers, and the shelving forms obstructions that prevents conditioned air being circulated evenly throughout the grow room. Even in non-tiered grow rooms, a thick plant canopy may interfere with proper circulation of ambient or conditioned air.

Temperature, humidity, carbon dioxide and plant surface temperatures are all factors that need to be controlled in indoor growing environments. These factors may be influenced by the quality of ambient air distribution inside a grow room. Having a quality air distribution breaks up stagnant air around the stomata (underside of leaves), promoting good carbon dioxide intake and proper transpiration. This problem of air quality inconsistency may be exacerbated in multi-tier grow rooms, because the tiers themselves interfere with even circulation of air, so extreme deviations in temperature, humidity, carbon dioxide levels and other environmental conditions are commonly found. For example, different air quality may exist in a grow room in the aisles, near the base of each plant and at the top of the grow room.

Currently, there are top-to-down style air flow systems where air is pulled in from the top and the air is delivered downward to the bases of the plants. However, these systems can cause harm to some plants in applications where grow lights are used, because the heat of the lights may be projected onto the plant canopy. Another problem with top-to-bottom air circulation systems is that they do a poor job at eliminating transpiration because the air movement caused by these systems is typically inadequate. Due to poor air movement, mold growth, powdery mildew, and bacterial may growth on the plants.

In single-tier grow rooms, traditional greenhouse fans can be used to move air above a plant canopy because there are generally no obstructions in the room to prevent air flow. However, these fans generally cannot maintain a consistent air quality throughout the room and, additionally, they are typically very large, taking up valuable indoor space.

As such, there is a need for an indoor airflow system that distributes conditioned air evenly throughout a grow room, at consistent volumes and air velocity, especially in multi-tier growing environments, while removing stagnant, hot, and humid air that may be detrimental to plant growth.

### SUMMARY

The present application describes a system, method and apparatus for creating laminar air flow inside an indoor growing environment. In one embodiment, an air distribution apparatus is described for creating a laminar air flow inside an indoor growing environment, comprising at least one fan casing comprising at least one electric fan for drawing ambient air proximate to the fan casing inside the fan casing, a horizontal air intake manifold coupled to a top portion of the at least one fan casing for receiving the ambient air from the at least one fan casing, the horizontal air intake manifold further comprising two or more air outlet ports, and two or more air tubes coupled to the two or more air outlet ports, respectively, each of the two or more air tubes extending between a particular row of the plurality of rows of plants, respectively, each of the two or more air tubes comprising a plurality of air tube orifices extending along each of the two or more air tubes that create a laminar flow of the ambient air as the ambient air is expelled through the plurality of air tube orifices, through a leaf canopy formed by the plurality of plants.

In another embodiment, a method is described for creating a laminar air flow for an indoor growing environment comprising a plurality of plants arranged in a plurality of rows, comprising drawing ambient air inside at least one fan casing, the fan casing comprising at least one electric fan, providing the ambient air from the at least one fan casing to an air intake plenum coupled to a top portion of the at least one fan casing, distributing the ambient air inside the air intake manifold to two or more air outlet ports of the air intake manifold, receiving, by two or more air tubes coupled to the two or more air outlet ports, respectively, the ambient air from the two or more air outlet ports, respectively, and discharging the ambient air from each of the two or more air tubes via a plurality of air tube orifices formed along a length of each of the two or more air tubes to create the laminar air flow through the plurality of plants.

### BRIEF DESCRIPTION OF THE DRAWINGS

The features, advantages, and objects of the present invention will become more apparent from the detailed description as set forth below, when taken in conjunction with the drawings in which like referenced characters identify correspondingly throughout, and wherein:

FIG. 1 is a perspective view of one embodiment of an air distribution apparatus for creating laminar air flow within an indoor growing environment;

FIG. 2 is a top, plan view of one of the air tubes of the air distribution apparatus as shown in FIG. 1;

FIG. 3 is a cutaway view of one of the air tubes of the air distribution apparatus as shown in FIG. 1 along a longitudinal axis of the air tube;

FIG. 4 is a perspective view of one embodiment of an air distribution system using the air distribution apparatus as shown in FIG. 1 plus an air exhaust assembly for creating laminar air flow within the indoor growing environment;

FIG. 5 is a perspective view of one embodiment of an air distribution system using the air distribution apparatus as shown in FIG. 1 plus the air exhaust assembly as shown in FIG. 4, wherein the air exhaust assembly additionally comprises an air plenum;

FIG. 6 is a perspective, exploded view of another embodiment of the air intake manifold as shown in FIGS. 1 and 4-5;

FIG. 7 is a bottom view of the air plenum as shown in FIG. 5;

FIG. 8 is a perspective view of another embodiment of the air distribution apparatus as shown in FIG. 1, mounted inside an indoor growing environment in an inverted position;

FIG. 9 is a flow diagram illustrating one embodiment of a method for creating a laminar air flow in an indoor growing environment; and

FIG. 10 is a side view of another embodiment of an air distribution system.

### DETAILED DESCRIPTION

Embodiments of the present invention are directed towards a system, method and apparatus for creating laminar air flow in indoor growing environments. “Laminar air flow”, as used herein, refers to a substantially uniform velocity and direction of air movement throughout an area inside an indoor growing environment where plants are located, which distributes conditioned air and air temperature evenly throughout the indoor growing environment. Such a laminar air flow promotes good plant growth and plant health. In one embodiment, a positive pressure zone is created under a plant canopy, delivering a laminar flow of ambient air upwards through the canopy. In another embodiment, a positive pressure zone is crated above a plant canopy, delivering a laminar flow of ambient air downwards through the canopy. In some embodiments, ambient air may be pulled from an upper, or lower, portion of an indoor growing environment, creating a negative pressure zone for removing moisture from transpiration of the plants and/or heat from any grow lamps.

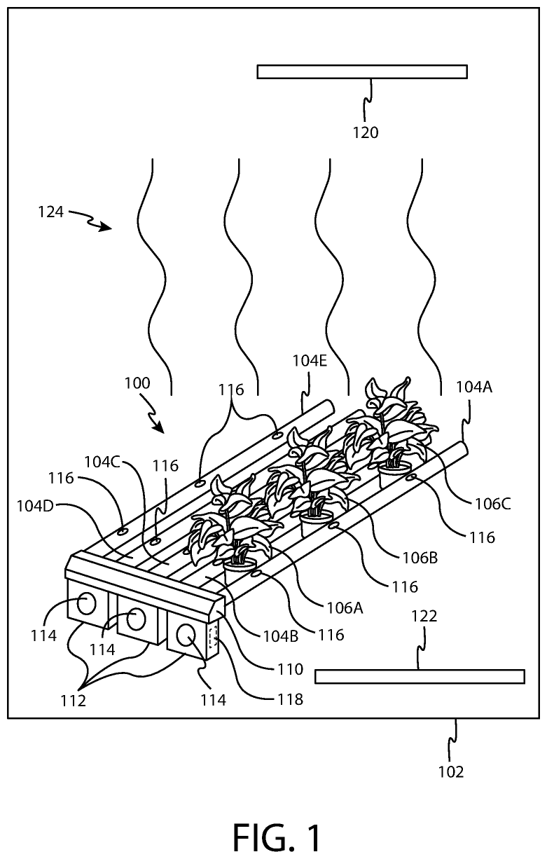

FIG. 1 is a perspective view of one embodiment of an air distribution apparatus 100 for creating laminar air flow 124 within indoor growing environment 102 and, in particular, through an area of indoor growing environment 102 where a plurality of plants 106 are located. Indoor growing environment 102 comprises a traditional outdoor greenhouse or one or more rooms inside a structure, such as a warehouse, a home, etc., typically measuring tens, or even hundreds, of feet in each dimension. For example, indoor growing environment 102 may comprise a greenhouse measuring 100 feet long by 70 feet wide and 14 feet tall. It should be understood that the relative dimensions of indoor growing environment 102 as shown in FIG. 1, as well as air distribution apparatus 100 and plants 106, are not to scale, either individually or in combination with each other.

Air distribution apparatus 100 comprises a horizontal air intake manifold 110, one or more fan casings 112 and a plurality of air tubes 104, in this example, five air tubes 104A-104E, extending horizontally from a rear side of air intake manifold 110. Ambient air proximate to the one or more fan casings 112, in a lower portion of indoor growing environment 102, is drawn into the one or more fan casings 112 and provided under pressure to air intake manifold 110. Each of the fan casings 112 comprises one or more electric fans 114, each sized to move a particular volume of air over a predetermined time through air distribution apparatus 100. For example, each fan 114 in each of the fan casings 112 may be rated to move 900 cubic feet per minute of ambient air. Although three fan casings 112 are shown coupled to air intake manifold 110, in other embodiments, fewer fan casings 112 are used and, in yet other embodiments, a greater number of fan casings 112 may be used. A length of air intake manifold 110 may increase or decrease to accommodate fewer or more fan casings.

A benefit of air distribution apparatus 100 is that it is modular in design, making it easy and affordable to add as many air distribution apparatus 100 as needed simply by placing two or more air distribution apparatus 100 side-by-side inside indoor growing environment 102.

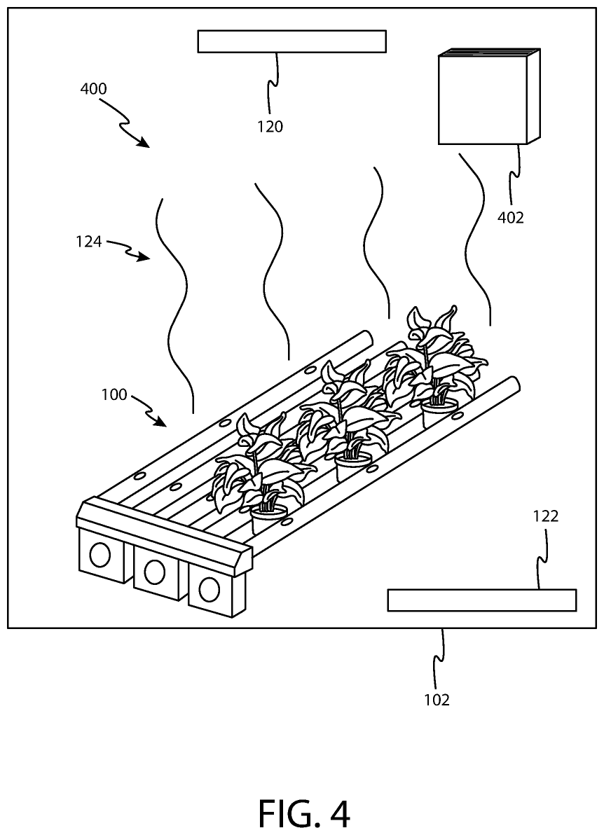

In the embodiment shown in FIG. 1, air intake manifold 110 is about 6½ feet long, 5½ inches wide, and 7 inches tall, while each fan casing 112 is approximately 16 inches square and a little more than 5 inches deep. These dimensions may vary based on the number of fan casings 112 used and the air movement rating of the electric fans in each fan casing 112. The components of air intake manifold 110 are typically formed of a hard and durable material, such as aluminum, steel, plastic, etc. A top portion of each fan casing 112 is mechanically coupled to air intake manifold 110 using well-known techniques, such as by welding, attachment with mechanical fasteners such as screws or rivets, by interlocking mechanical tabs/slots, or by some other means. The top portion of each fan casing 112 is open, allowing ambient air to flow from each fan casing 112 into air intake manifold 110, better shown in FIG. 4.

In some embodiments, one or more air sanitizers 118 are used to sanitize the ambient air. In one embodiment, one or more air sanitizers 118 are located inside one or more of the fan casings 112 as shown. In other embodiments, additionally or alternatively, one or more air sanitizers 118 may be located inside air intake manifold 110. As ambient air is pulled into the fan casings 112 and forced into air intake manifold 110, the one or more air sanitizers 118 condition the ambient air to kill mold particles and bacteria and may prevent mold particles and bacteria from attaching to the plants. In one embodiment, at least some of the air sanitizers 118 comprise a bipolar ionizer.

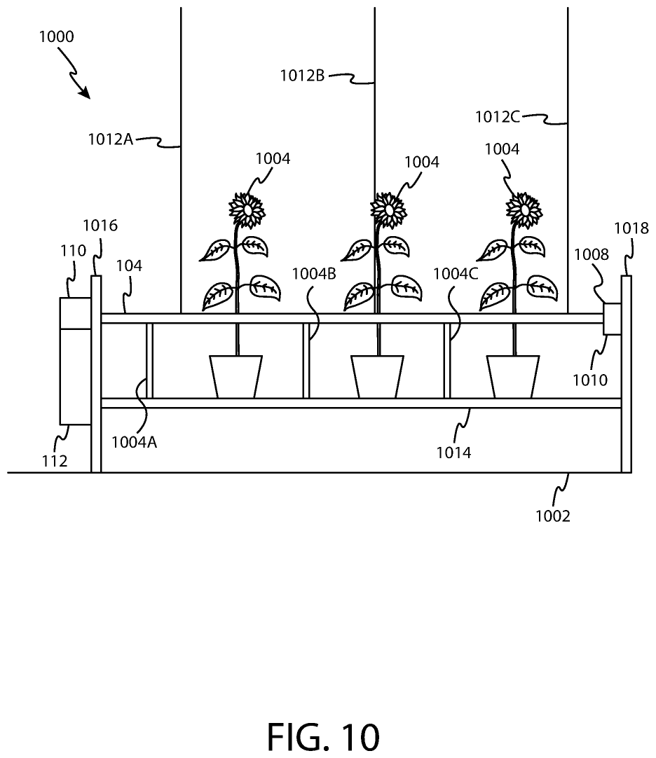

As mentioned above, ambient air in proximity to air intake manifold 110 is drawn into air intake manifold 110 by the electric fan(s) in each fan casing 112 and then provided to air intake manifold under pressure. Air intake manifold 110 is sized and shaped to distribute the ambient air substantially evenly along a length of air intake manifold 110. The ambient air inside is then expelled through two or more air outlet ports (not shown in this view) on a rear side of air intake manifold 110. FIG. 1 illustrates an embodiment where five such air tubes are used, 104A through 104E. In other embodiments, a fewer or a greater number of air tubes can be used per air intake manifold 110. In some embodiments, as shown in FIG. 1, the air tubes 104 are placed on the ground of indoor growing environment 102 on each side of a row of plants (coupled to the air outlet ports of air intake manifold 110 via flexible hoses, not shown), while in other embodiments, such as the embodiment shown in FIG. 10, the air tubes 104 are raised off of the ground. Generally, the longer air intake manifold 110, the greater number of air tubes 104 may be accommodated. Generally, the total number of air tubes 104 in indoor growing environment 102 is selected based on the number of rows of plants inside indoor growing environment 102. Ideally, the air tubes 104 are placed between each row of plants. Additionally, the total number of air tubes needed may depend on a width of each row of plants, i.e., the greater the width of each row, the fewer total number of air tubes 104 may be needed. When many air tubes 104 are needed, two or more air intake manifold 110 may be installed side-by-side, each air intake manifold 110 comprising, for example, five air tubes 104. The modular design of air distribution apparatus 100 allows for such tailored expansion.

Each of the air tubes 104 may extend a length approximately equal to a length of each row of plants, such the length of a row of plants formed by plants 106A-106C, as shown in FIG. 1. The air tubes 104 need not be equal in length. For clarity, only three plants 106 are shown in FIG. 1 so as to not obscure air tubes 104C-104E, and the plants are not shown in proportion to air distribution apparatus 100 or indoor growing environment 102. In practice, the number of plants in each row could be 50 plants or more, generally limited by a size of the plants and a size of indoor growing environment 102.

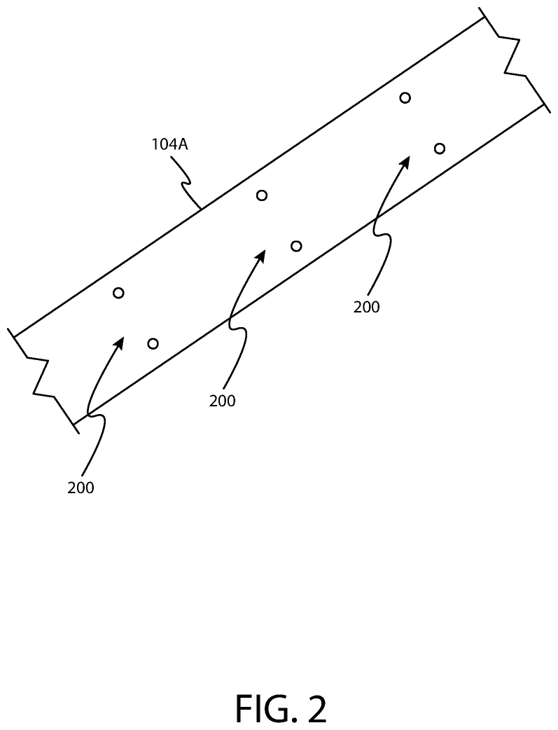

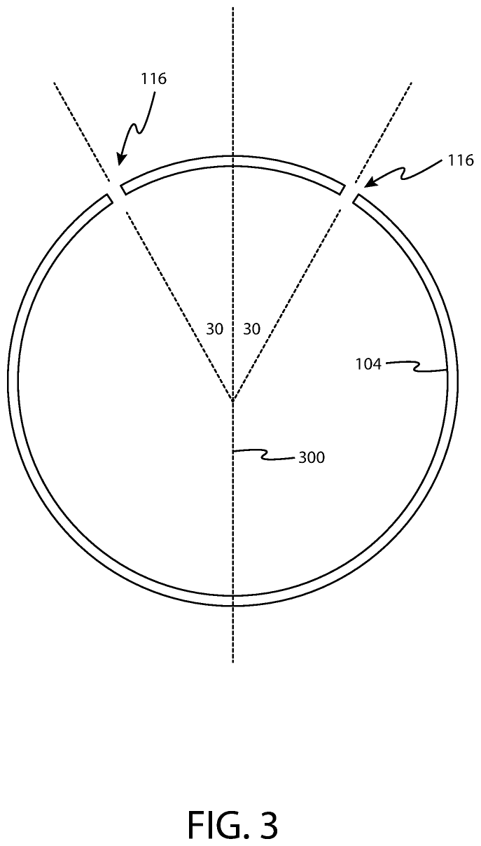

Each of the air tubes 104 are sealed at a distal end, forcing pressurized air from air intake manifold 110 to be expelled through a plurality of air tube orifices 116 extending along a length of each of the air tubes 104. The pressurized ambient air flowing through the air tube orifices 116 form a laminar air flow throughout an area of indoor growing environment 102 where plants are located. In the embodiment shown in FIG. 1, multiple pairs of air tube orifices 116 are spaced apart along the length of each air tube 104 at a predetermined angle from the vertical. This relationship is better shown in FIGS. 2 and 3.

FIG. 2 is a top, plan view of one of the air tubes 104 as shown in FIG. 1, while FIG. 3 is a cutaway view of the same air tubes along a longitudinal axis of the air tube. FIG. 2 shows three sets of air tube orifices 200 spaced apart from each other, each set 200 comprising two air tube orifices 116 spaced apart from each other at about 30 degrees from each other from imaginary vertical axis 300. The angle at which the air tube orifices 116 are spaced apart from each other may vary depending on factors such as plant age, plant height, the number of air tube orifices 116 in each air tube 104, etc. In generally, the amount of separation is selected to direct the expelled air from the air tube orifices 116 towards plants on either side of each are tube 104. The predetermined angle typically ranges between 45 degrees and 5 degrees. In other embodiments, a single orifice is formed in place of each pair of air tube orifices, aligned substantially to the vertical. In yet other embodiments, three or more air tube orifices can be formed at predetermined spacings along each air tube 104, for example, one directed substantially in the vertical, and to others at a predetermined angle from the vertical, such as 10 degrees. In still yet other embodiments, air tube orifices 116 may be formed uniformly throughout a top portion of each air tube 104, such as 12 air tube orifices per foot covering a top surface of each air tube 104 from −20 degrees to +20 degrees from the vertical. Other arrangements of air tube orifices are also contemplated.

Ambient air from air intake manifold 110 is generally pressurized, as the airflow rate from the electric fans in the fan casings 112 is generally greater than the total air flow rate that can escape from the plurality of air tube orifices 116. The number and diameter of the air tube orifices, plus the number of air tubes 104 and the total air flow volume from the fans in the fan casings 112, determines the pressure inside of the air tubes 104 and a laminar air flow rate through the plants. For example, in one embodiment, the air tube orifices 116 are spaced about 2 feet from each other along the length of each air tube 104, comprising a diameter each of approximately ⅝ inch. Assuming a total air flow rate of, for example, 3,000 cubic feet per minute through all of the air tube orifices 116, this creates a pressure of about between 0.9 and 1.3 inches of water column inside each of the air tubes 104 and a laminar airflow rate of about between 540 and 900 cubic feet per minute of ambient air flow per air tube 104 upwards through the plants or approximately 9-15 cubic feet per minute per square foot of coverage area, for example, a length of the air tubes 104 multiplied by the width of air intake manifold 110, in this embodiment, about 225 square feet.

The distance between the air tube orifices 116 may be increased or decreased to create a same air flow volume regardless of the length of each air tube 104 at an ideal velocity. For example, if the length of each air tube 104 is increased by a factor of 2, then the spacing between the air tube orifices 116 may be increased by a factor of 2 to maintain a same laminar flow rate through the plants. Similarly, if the length of the air tubes 104 is reduced by a factor of 2, the distance between the air tube orifices 116 may be decreased by a factor of 2 in order to maintain the same laminar flow rate.

Each air tube 104 is formed of a rigid, flexible or semi-flexible material that may allow each are tube 104 to bend, for example, plus or minus 30 degrees or more, in case the rows of plants are not aligned in a straight line or not aligned in rows at all. The material typically allows the air tubes 104 to be cut on-site to a length matching a length of each row of plants. In one embodiment, each air tube 104 is 35 feet long, 4 inches in diameter and approximately ⅛ inch in thickness and formed of polyduct. The thickness of each air tube 104 is selected to ensure mechanical integrity of each are tube 104 given an expected air pressure inside each are tube 104, for example, and air pressure between 0.9 and 1.3 inches of water column.

As ambient air is forced upwards through the plants from the air orifices 116, an overall positive air pressure is created in a lower portion of indoor growing environment 102, generally underneath a canopy of leaves created by the plants, pushing the ambient air upwards, through the plant canopy, towards an upper portion of indoor growing environment 102. The ambient air typically picks up moisture that is transpiring from the plants as well has heat from indoor grow lights. Additionally, bacteria and mold may be picked up as well. The ambient air, thus, may become “contaminated” with such undesirable elements. The contaminated air in the upper portion of indoor growing environment 102 may be removed by an HVAC system (not shown) via return vent 120. Indoor growing environment 102 typically comprises an HVAC system, and the contaminated air is removed from the upper portion of indoor growing environment 102 via return vent 120 and returned to a bottom portion of indoor growing environment 102 via supply vent 122 to supply ambient air to air intake manifold 110. The HVAC system may additionally condition the ambient air, i.e., it may heat or cool the ambient air, dehumidify or add humidity to the contaminated air, etc., producing semi-conditioned or conditioned air back to a lower area of indoor growing environment 102. At this point, the air may be referred to as “ambient air” once again.

There are several benefits to creating a laminar airflow by air distribution apparatus 100 upwards through the plants, as described above. For example, creating a uniform air flow throughout indoor growing environment 102 or a portion thereof where plants are located, breaks up stagnant, hot, humid air around the stomata of the plants. This airflow helps to regulate various growing conditions such as temperature, humidity, carbon dioxide and plant surface temperatures. In some embodiments, air distribution apparatus 100 additionally sanitizes the air so that mold and bacteria formation is reduced or eliminated. An additional benefit of using air distribution apparatus 100 is that it may aid the performance and efficiency of an HVAC system that services indoor growing environment 102.

FIG. 4 is a perspective view of one embodiment of an air distribution system 400 for creating laminar air flow 124 within indoor growing environment 102. Shown is air distribution apparatus 100 and air exhaust assembly 402. Air distribution apparatus 100 operates as previously described, creating a positive air pressure in a lower portion of indoor growing environment 102, thereby creating a laminar air flow upwards through the plants and plant canopy. The ambient air expelled by the air tubes 104 rises to an upper portion of indoor growing environment 102, becoming “contaminated”. Air exhaust assembly 402 comprises a housing and one or more internal electric fans or other air movement device(s). The contaminated air in the upper portion of indoor growing environment 102 is drawn inside air exhaust assembly 402, which creates a negative air pressure in the upper portion of indoor growing environment 102. The contaminated air is then expelled from air exhaust assembly 402 to move the contaminated air away from the upper portion of indoor growing environment 102, either venting the contaminated air outside indoor growing environment 102 or providing the contaminated air to an HVAC system via return vent 120. The contaminated air may then be conditioned by the HVAC system, or not, and returned to a lower portion of indoor growing environment 102 via supply vent 122. Using both air distribution device 100 to create a positive pressure in the lower portion of indoor growing environment 102 and air exhaust assembly 402 to create a negative pressure in the upper portion of indoor growing environment 102 creates an “air exchange”, as opposed to just delivering air and letting it mix with stagnant air around the plant canopy. Air exhaust assembly 402 may be selected to exchange all of the air in the upper portion of indoor growing environment 102 quickly, for example, given a canopy height of six feet, at a rate of between 20 seconds and 60 seconds, at a rate proportional to the laminar flow rate produced by the air tubes 104 per square foot of coverage area, or canopy area. It should be understood that although only one air exhaust assembly 402 is shown in FIG. 4, in other embodiments, two or more air exhaust assemblies 402 may be used to remove contaminated air from the upper portion of indoor growing environment 102.

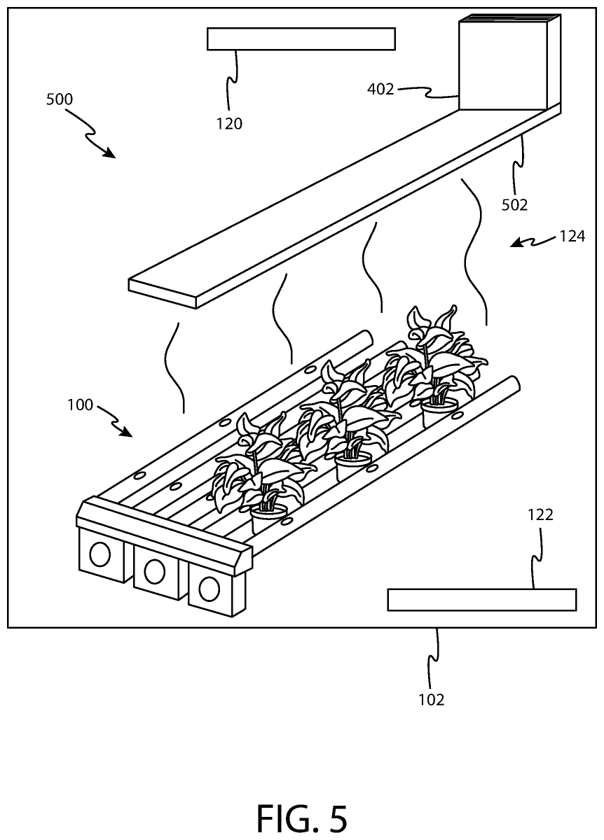

FIG. 5 is a perspective view of another embodiment of an air distribution system 500 for creating laminar air flow 124 within indoor growing environment 102. Shown is air distribution apparatus 100 and air exhaust assembly 402, with air exhaust assembly 402 further comprising air plenum 502. Air distribution apparatus 100 operates as previously described, creating a positive air pressure in a lower portion of indoor growing environment 102, thereby creating a laminar air flow upwards through the plants. Air plenum 502 is coupled to air exhaust assembly 402, comprising a relatively thin, hollow air duct extending generally horizontally from air exhaust assembly 402 over the plants and into the upper portion of indoor growing environment 102. Air plenum 502 draws the contaminated air in the upper portion of indoor growing environment 102 through a plurality of plenum orifices (not shown in this view) on a bottom surface of air plenum 502 as a result of negative air pressure created by air exhaust assembly 402. Air plenum 502 extends the negative pressure over the plants and allows the contaminated air in the upper portion of indoor growing environment 102 to be more evenly drawn away from an area above the plant canopy. Once inside air plenum 502, the contaminated air is directed towards air exhaust assembly 402, where it is expelled outside of indoor growing environment 102 or provided to HVAC equipment via return vent 120. The contaminated air may then be returned to a lower portion of indoor growing environment 102 via supply vent 122 as “ambient air”. It should be understood that although only one air exhaust assembly 402 and air plenum 502 is shown in FIG. 5, in other embodiments, two or more air exhaust assemblies 402, each with an associated air plenum 502, may be used to remove ambient air from the upper portion of indoor growing environment 102. In some embodiments, a combination of air exhaust assemblies 402 may be used, some with air plenum 502 and some without.

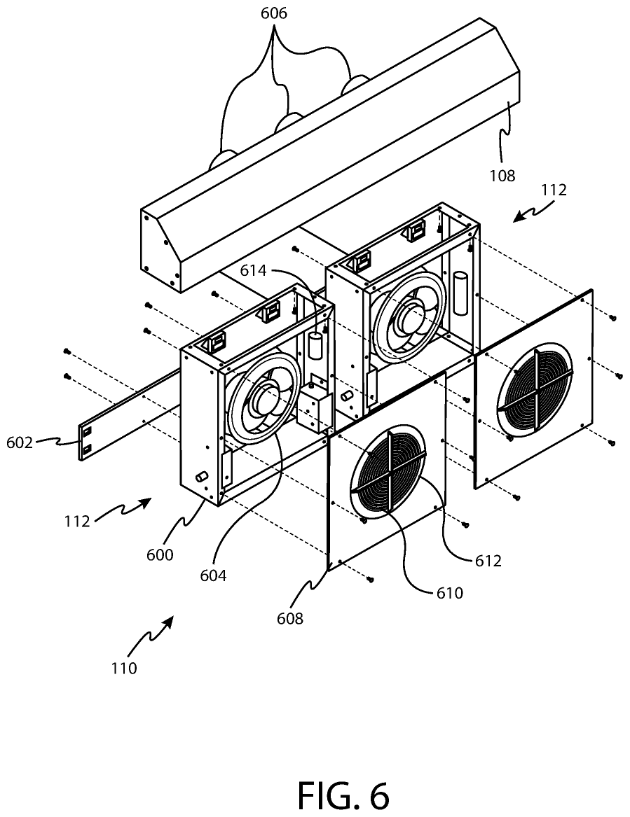

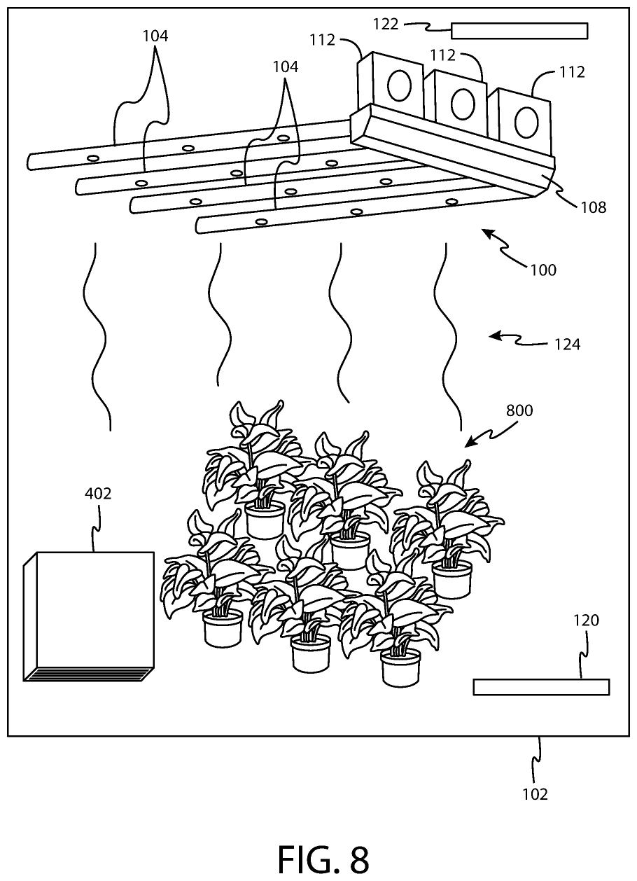

FIG. 6 is a perspective, exploded view of another embodiment of air intake manifold 110, with only two fan casings 112. Each fan casing 112 comprises a housing 600 mechanically coupled to a rack mount 602. Rack mount 602, in this embodiment, comprises a single, horizontal, rigid panel that attaches to existing rack equipment in indoor growing environment 102. In other embodiments, rack mount 602 may comprise one or more other mechanical means to mount air intake manifold 110 in a desired position within indoor growing environment 102. Rack mount 602 allows air intake manifold 110 to be mounted to racks inside indoor growing environment 102 either in an upright position, as shown in FIGS. 1 and 4-5, or in an inverted position, as shown in FIG. 8.

Each fan casing 112 additionally comprises one or more electric fans 604 mounted to housing 600, for drawing ambient air proximate to air intake manifold 110 inside housing 600 and expelling it proportionally through two or more air outlet ports 606, and into two or more air tubes 104, respectively. Fan 604 may comprise a centrifugal fan, a plenum fan, an inline fan, or the like. In some embodiments, a backward-curved blade fan is used. Typically, fan 604 is selected based on its ability to move air over a predetermined time period as well as its size, power consumption and cost. Preferably, fan 604 is slim in depth, no wider than the depth of air intake manifold 110, in this example, no deeper than 5½ inches, so air intake manifold 110 can be installed at the end of a rack inside indoor growing environment 102, preferably at a backside of a rack where there is typically minimal space between the rack and a wall of indoor growing environment 102. Each fan is typically adjustable via a control switch (not shown) to provide a range of airflow rates, for example, an airflow range of between 500 cubic feet per minute to 2,000 cubic feet per minute. A housing cover 608 encloses electric fan 604 inside housing 600 and allows ambient air to enter housing 600 through a hole formed in housing cover 608. A fan grille 610 covers the hole, and in some embodiments is mounted to a flange 612 mounted to the hole.

In some embodiments, sanitizer 118 is located inside one or more of the fan casings 112 and/or inside air intake manifold 110 for conditioning the ambient air to kill mold and bacteria. FIG. 6 illustrates sanitizer 118 mounted to each of the fan casings 112. In one embodiment, sanitizer 118 comprises a bipolar ionizer, but in other embodiments, in addition or alternative to a bipolar ionizer, sanitizer 118 may comprise an air filter, one or more UV lights, and/or a dehumidifier.

When assembled, ambient air proximate to air distribution apparatus 110 is drawn into housing 600, forced into air intake manifold 110 and expelled out through the two or more air outlet ports 606 and into two or more air tubes 104, respectively. Typically, a positive pressure is created inside air intake manifold 110, as the ambient air being expelled into the air tubes 104 cannot freely escape the air tubes 104, due to the limited number and size of the air tube orifices.

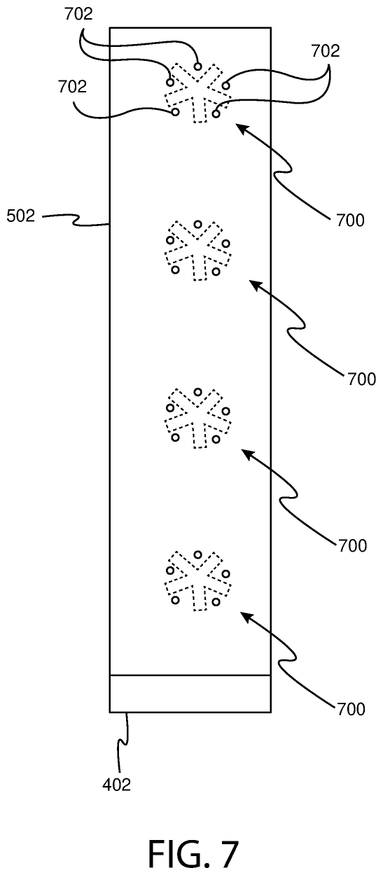

FIG. 7 is a bottom view of air exhaust assembly 402 and air plenum 502 as shown in FIG. 5. It should be understood that air exhaust assembly 402 and air plenum 502 are not drawn to scale. Typically, air plenum 502 extends tens of feet away from air exhaust assembly 402. FIG. 7 shows four air intake sections 700, each section 700 comprising a plurality of air intake orifices 702 and an adjustable baffle 704. Each section 700 allows contaminated air in an upper portion of indoor growing environment 102 to be drawn into air plenum 502 via the air intake orifices 702. While each section 700 shown in FIG. 7 comprises five air intake orifices 702, in other embodiments, fewer or a greater number of air intake orifices 702 could be used, and the diameter of each air intake orifices five or two may be greater or smaller than depicted in FIG. 7. Adjustable baffle 704 may be located inside or external to air plenum 502, comprising, in this embodiment, five “arms”, each arm for completely or partially covering a respective air intake orifices 702 as baffle 704 is rotated in place by a user.

FIG. 8 is a perspective view of another embodiment of air distribution apparatus 100 mounted inside indoor growing environment 102 in an inverted position. In this embodiment, air distribution apparatus 100 is inverted from the position shown in FIG. 1, where air intake manifold 110 is secured to one or more racks or other support means inside indoor growing environment 102. The racks or other support means are not shown in order to highlight the features of air distribution apparatus 100. In this embodiment, only four air tubes 104 are used instead of the five shown in FIG. 1.

In the position shown in FIG. 8, ambient air in proximity to air intake manifold 110 in an upper portion of indoor growing environment 102 is drawn into the fan casings 112, into air intake manifold 110 and expelled into four air outlet ports 606 (hidden from view). The four air tubes 104 are coupled to the air outlet ports and distribute the ambient air evenly throughout indoor growing environment 102 via the plurality of air tube orifices 116, pointing downward, thus creating a laminar airflow in a downward direction, towards plants 800. In one embodiment, air exhaust assembly 402 may be used to move the air that has collected in a lower portion of indoor growing environment 102 and may have become contaminated by the moisture from the plants, heat from grow lamps, mold, bacteria, etc. Air exhaust assembly 402 provides the contaminated air to an HVAC system via return vent 120. The contaminated air may then be conditioned by the HVAC system and circulated back to the upper portion of the indoor growing environment 102 via supply vent 122. In other embodiments, air exhaust assembly 402 is not used, in embodiments where the HVAC system is able to remove the contaminated air from the lower portion of indoor growing environment 102 at a rate that maintains the laminar flow of ambient air through the plants 800.

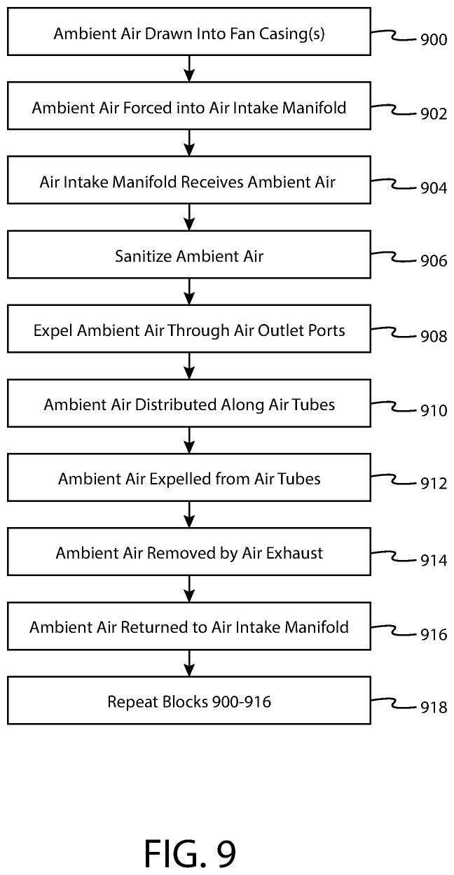

FIG. 9 is a flow diagram of a method for creating laminar air flow in an indoor growing environment. It should be understood that in some embodiments, not all of the steps shown in FIG. 9 is performed and that the order in which the steps are carried out may be different in other embodiments. It should be further understood that some minor method steps may have been omitted for purposes of clarity.

At block 900, ambient air in proximity to air intake manifold 110 is drawn into one or more fan casings 112. Each fan casing comprises an electric fan that draws the ambient air inside each fan casing.

At block 902, the ambient air is forced into air intake manifold 110 from each of the fan casings 112.

At block 904, air intake manifold 110 receives the forced air from fan casings 112.

At block 906, one or more air sanitizers 118 located inside one or more of fan casings 112 and/or inside air intake manifold 110 may be used to sanitize the ambient air as the ambient air passes from fan casings 112 to two or more air outlet ports 406.

At block 908, the forced ambient air is expelled from air intake manifold 110 through the two or more air outlet ports 406.

At block 910, the forced ambient air is distributed among two or more air tubes 104, each of the air tubes 104 coupled to a respective air outlet port 406, each of the air tubes 104 spanning a length of indoor growing environment 102 in between rows of a plurality of plants 600 near their base.

At block 912, the forced ambient air inside each of the air tubes 104 is expelled through a plurality of air tube orifices 116, creating a substantially laminar air flow throughout indoor growing environment 102. In the embodiment shown in FIGS. 1-3, the laminar airflow moves in an upward direction against the stomata of the plants 600. In the embodiment shown in FIG. 6, the laminar airflow moves in a downward direction against the cuticle of the leaves of plants 600.

At block 914, in some embodiments, air exhaust assembly 402 removes contaminated air proximate to air exhaust assembly 402 and provides the contaminated air to either an HVAC system via return vent 120 or vents the contaminated air to outside of indoor growing environment 102. This typically creates a negative pressure in an area of indoor growing environment 102 proximate to air exhaust assembly 402. In some embodiments, as shown in FIG. 3, an air plenum 302 extends substantially horizontally from air exhaust assembly 402, removing ambient air in proximity to air plenum 302 and feeding it to air exhaust assembly 202.

At block 916, in one embodiment, the contaminated air is conditioned by the HVAC system and is returned to an area proximate air intake manifold 110 via supply vent 122.

At block 918, this process repeats, with the ambient air in proximity to air intake manifold 110 drawn into one or more fan casings 112.

FIG. 10 is a side view of another embodiment of an air distribution apparatus 1000, similar to the embodiment shown in FIG. 1, except that the air tubes 104 are raised off of floor 1002 of indoor growing environment 102 in order to deliver a laminar air flow closer to a canopy of leaves formed by the plants 1004. Raising the air tubes 104 further towards a canopy of leaves formed by the plants 1004 may be beneficial to plant health for certain types of plants that, for example, are tall in nature or which have a canopy that is concentrated towards an upper portion of indoor growing environment 102. It should be understood that the relative dimensions of indoor growing environment 102 as shown in FIG. 10, as well as air distribution apparatus 1000 and plants 106, are not to scale, either individually or in combination with each other. It should be further understood that only one air tube 104 is shown, as one or more other air tubes are hidden behind the one shown in FIG. 10.

As shown, air intake manifold 110, along with one or more fan casings 112, is suspended off of floor 1002 using an object placed beneath the fan casing(s) (not shown) or by attachment to one or more front support members 1016 within indoor growing environment 102. One or more benches 1014 extend from a lower portion of front support member, fastened to rear support member 1018 at a lower portion of rear support member 1018. The one or more benches 1014, together with front support members 1016 and rear support member 1018, form a “rack” that supports air intake manifold 110, the plants 1004 and the air tubes 104, as explained below.

Each of the air tubes 104 extending from air intake manifold 110 is supported, in one embodiment, at a distal end of each air tube 104 by a mechanical coupling 1008 to an upper portion of rear support member 1018. In other embodiments, one or more lower supports 1004A-1004C are used to support the air tubes 104 off of floor 1002, typically extending from a top surface of the one or more boards 1014. In yet other embodiments, one or more upper supports 1012A-1012C are used to support the air tubes 104 from an upper portion of indoor growing environment 102, such as from the ceiling, beams, lights, etc. of indoor growing environment 102. In some embodiments, one or all of the mechanical coupling, lower supports 1004 and the upper supports are used. Although only 3 lower supports 1004 and upper supports 1012 are shown, in practice, five or more mechanical supports are typically used and, in general, increasing numbers as the length of the air tubes 104 increase. Mechanical coupling 1008 comprises any mechanical assembly that can be mechanically fixed to rear support member 1018 and attach to the ends of the air tubes 104, such as a metal plate, retaining coupling, a “pole socket”, etc. Mechanical coupling 1008 may additionally be used to seal the distal end of each air tube 104, such as by insertion of the distal end into mechanical coupling 1008 and securing it thereto via a hose clamp. Lower supports 1004 comprise any rigid material such as wood, plastic, etc., each with an area cross-section large enough to support a corresponding portion of a weight of each air tube 104. The upper supports 1012 may comprise rigid material such as wood, plastic, etc., or they may be formed from wire, rope, or some other similar material.

Throughout the description and drawings, example embodiments are given with reference to specific configurations. It will be appreciated by those of ordinary skill in the art that the present invention can be embodied in other specific forms. Those of ordinary skill in the art would be able to practice such other embodiments without undue experimentation. The scope of the present invention, for the purpose of the present patent document, is not limited merely to the specific example embodiments or alternatives of the foregoing description.

I claim:

1. An air distribution apparatus for an indoor growing environment comprising a plurality of plants arranged in a plurality of rows, comprising:
at least one fan casing comprising at least one electric fan for drawing ambient air proximate to the fan casing inside the fan casing;
a horizontal air intake manifold coupled to a top portion of the at least one fan casing for receiving the ambient air from the at least one fan casing, the horizontal air intake manifold further comprising two or more air outlet ports; and
two or more air tubes coupled to the two or more air outlet ports, respectively, each of the two or more air tubes extending between a particular row of the plurality of rows of plants, respectively, each of the two or more air tubes comprising a plurality of air tube orifices extending along each of the two or more air tubes that create a laminar flow of the ambient air as the ambient air is expelled through the plurality of air tube orifices, through a leaf canopy formed by the plurality of plants.

2. The air distribution apparatus of claim 1, further comprising:
an air exhaust assembly mounted separately from the air distribution apparatus, for drawing in contaminated air from an upper area of the indoor growing environment, the ambient air becoming contaminated after being propelled through the leaf canopy, and providing the contaminated air to an air conditioning return vent inside the indoor growing environment.

3. The air distribution apparatus of claim 2, further comprising:
an air plenum coupled to the air exhaust assembly and extending substantially horizontally therefrom over the plant canopy, the air plenum comprising a plurality of air plenum orifices for allowing the contaminated air in the upper area of the indoor growing environment to enter the air plenum and feed the contaminated air inside the air plenum to the air exhaust assembly.

4. The air distribution apparatus of claim 1, further comprising:
an air sanitizer for sanitizing the ambient air.

5. The air distribution apparatus of claim 4, wherein the air sanitizer is mounted within the air intake manifold.

6. The air distribution apparatus of claim 5, wherein the air sanitizer comprises a bipolar ionizer.

7. The air distribution apparatus of claim 1, wherein the plurality of air tube orifices is spaced evenly apart from each other along each of the two or more air tubes, respectively.

8. The air distribution apparatus of claim 1, wherein the plurality of air tube orifices is arranged in pairs along each of the two or more air tubes, each one of the air tube orifices offset from one another on opposing sides of a longitudinal center of each one of the air tubes, respectively, for directing the ambient air towards a first row of the plurality of rows of plants and a second row of the plurality of rows, respectively.

9. The air distribution apparatus of claim 1, wherein the intake manifold is configured for operation in a first upright position, wherein the laminar flow of the ambient air is directed upwards through an underside of the plant canopy to an upper area of the indoor growing environment, or for operation in a second, inverted position, wherein the laminar flow of the ambient air is directed downwards from the upper area in the indoor growing environment through the plant canopy to a lower area in the indoor growing environment.

10. The air distribution apparatus of claim 1, wherein the plurality of air tube orifices is sized relative to a number of the plurality of air tube orifices and an air flow rate of the at least one electric fan.

11. A method for creating a laminar air flow for an indoor growing environment comprising a plurality of plants arranged in a plurality of rows, comprising:
drawing ambient air inside at least one fan casing, the fan casing comprising at least one electric fan;
providing the ambient air from the at least one fan casing to an air intake plenum coupled to a top portion of the at least one fan casing;
distributing the ambient air inside the air intake manifold to two or more air outlet ports of the air intake manifold;
receiving, by two or more air tubes coupled to the two or more air outlet ports, respectively, the ambient air from the two or more air outlet ports, respectively; and
discharging the ambient air from each of the two or more air tubes via a plurality of air tube orifices formed along a length of each of the two or more air tubes to create the laminar air flow through the plurality of plants.

12. The method of claim 11, further comprising:
drawing in contaminated air from an upper area of the indoor growing environment by a separate air exhaust assembly, the ambient air becoming contaminated after being propelled through the leaf canopy, and providing the contaminated air to an air conditioning return vent inside the indoor growing environment.

13. The method of claim 12, wherein drawing the contaminated air from the plurality of air tube orifices in an upper area of the indoor growing environment comprises:
drawing the contaminated air through an air plenum coupled to the air exhaust assembly and extending substantially horizontally therefrom over the plant canopy, the contaminated air drawn into the air plenum via a plurality of air plenum orifices in the air plenum and feeding the contaminated air inside the air plenum to the air exhaust assembly.

14. The method of claim 11, further comprising:
sanitizing the ambient air with an air sanitizer.

15. The method of claim 14, wherein the air sanitizer is located inside the air intake manifold.

16. The method of claim 15, wherein the air sanitizer comprises a bipolar ionizer.

17. The method of claim 11, wherein the plurality of air tube orifices is spaced evenly apart from each other along each of the two or more air tubes, respectively.

18. The method of claim 11, wherein the plurality of air tube orifices is arranged in pairs along each of the two or more air tubes, each one of the air tube orifices offset from one another on opposing sides of a longitudinal center of each one of the air tubes, respectively, for directing the ambient air towards a first row of the plurality of rows of plants and a second row of the plurality of rows, respectively.

19. The method of claim 11, wherein the intake manifold is configured for operation in a first upright position, wherein the laminar flow of the ambient air is directed upwards through an underside of leaves of the plurality of plants to an upper area of the indoor growing environment, and for operation in a second, inverted position, wherein the laminar flow of the ambient air is directed downwards from the upper area in the indoor growing environment through a topside of the leaves of the plurality of plants to a lower area in the indoor growing environment.

20. The method of claim 11, wherein the plurality of air tube orifices is sized relative to a number of the plurality of air tube orifices and an air flow rate of the at least one electric fan.
